(19) United States
(12) Patent Application Publication
(10) Pub. No.: US 2005/0177057 A1
(43) Pub. Date: Aug. 11, 2005

(54) Automated breath collection device

(76) Inventors: Mitchell Friedman, Baltimore, MD (US); Peter Klein, Houston, TX (US); E. Roseland Klein, Fort Davis, TX (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 10/772,833

(22) Filed: Feb. 5, 2004

(51) Int. Cl. B65D 81/00; A61B 5/08

(52) U.S. Cl. 600/543

(57) ABSTRACT

An automated device for collecting breath samples, which includes pre-programmed protocols for specified breath collection procedures and pre-programmed voice chips connected to a speaker that audibly guide a patient or subject through the breath collection process. The device includes an inlet and a breath reservoir for collecting a sample before diverting it to a vessel via a valve manifold with a plurality of valves, each associated with breath collection vessel.

### FIELD OF THE INVENTION

This invention relates to a device for collecting a breath sample from a subject. More specifically, it relates to an automated device for collecting and retaining one or more breath samples from a subject.

### BACKGROUND

A number of diagnostic procedures or physiological assessments involve the measurement of metabolites or other substances in the breath of a subject. One such diagnostic method commercially available worldwide uses orally administered stable isotope labeled substrates. Typically, carbon-13 (¹³C) is the stable isotope used. Carbon-12 is the predominate form of carbon on earth and the majority of carbon exhaled in breath is carbon-12. The presence of ¹³C (derived from a labeled substrate) in the breath can thus be used to assess the presence of disease and metabolic dysfunction involving the metabolism of a carbon substrate.

When administered orally or intravenously, the labeled substrate is metabolized by various means in the body resulting in any number of byproducts, including, ¹³CO₂. The ¹³CO₂ so produced is absorbed by the blood and ultimately expired through the nose and/or mouth during respiration resulting in a higher concentration of ¹³CO₂ in the breath when compared to a pre-administration sample of breath. A change (or lack thereof) in the ¹³CO₂:¹²CO₂ ratio expired in post-administration exhalations (relative to pre-administered exhalations) is diagnostically valuable.

Certain disease states or metabolic dysfunctions can be assessed by the detecting the change or lack of change of the ¹³CO₂:¹²CO₂ ratio. For example, gastrointestinal infection by Helicobacter pylori may be detected by taking advantage of the presence of urease in its cell coat, an enzyme that hydrolyzes urea forming carbon dioxide and ammonium. If present, the bacteria will hydrolyze an orally administered dose of labeled urea and produce labeled CO₂. The labeled CO₂ is absorbed through the stomach wall into the blood and excreted in the lungs. If a ¹³C label is utilized, ¹³CO₂ can be detected by a breath test.

Similarly, breath tests can be employed to assess dysfunction in gastric emptying. To assess gastric emptying rate, a patient ingests a meal labeled with ¹³C. As the meal is absorbed and metabolized, ¹³CO₂ is produced as a byproduct of metabolism, absorbed into the blood and expired through the breath. Successive post-meal breath samples are collected over a prescribed time. The amount ¹³CO₂ excreted in the breath is plotted against time to create an excretion curve from which the rate of gastric emptying can be determined.

Finally, the diagnosis of certain inherited metabolic disorders is amendable to the breath test platform. A specific example is the ¹³C-galactose breath test used to assess whole body galactose oxidation in children and newborns suspected to have inherited galactosemia, a genetic metabolic disorder that is highly amenable to treatment. Diagnosis is made by quantification of whole body oxidation. Whole body oxidation can be quantified by orally administering a bolus of labeled galactose. If ¹³C label is used then the determination of the amount of ¹³CO₂ post administration is diagnostically valuable. A rise in post administration ¹³CO₂ indicates normal oxidation. Little or no rise is indicative of the inherited disorder of galactosemia.

Typically, the measurement of ¹³CO₂:¹²CO₂ isotope ratios is carried out by gas-isotope-ratio mass spectrometry (GIRMS). GIRMS is a preferred analytical method because small sample volumes combined with high instrument precision and sensitivity yield highly accurate results. Additionally, non-dispersive infrared spectrophotometers may be utilized. However, infrared spectrophotometry is less desirable because larger sample volumes are required and the instrumentation yields lower precision.

Regardless of the analytical method employed to determine isotopic ratios, breath samples must be collected and preserved for analytical testing. A number of collection protocols are known. Typically, patients exhale a breath sample into a balloon or breath bag. The sample is then transferred to an airtight container such as the pre-evacuated tube sold by Becton & Dickinson under the trademark Vacutainer®. This multi-step process is inconvenient and risks loss or dilution of the sample during the transfer process.

Recently, the use of breath analyzers into which a breath sample is directly delivered has been proposed. For example, U.S. Pat. No. 6,186,958 describes a device that includes an infrared spectrophotometer. The patient exhales into the device and the sample is instantly analyzed. However, this device provides no means to capture and retain the breath sample, rather, after the sample is analyzed it is purged from the device so that the next sample may be taken and analyzed.

It would be desirable to provide a means whereby breath samples can be easily collected and analyzed with all or part of the sample being captured for storage.

### SUMMARY OF THE INVENTION

According to the present invention, an automated device for collecting samples of breath expired from a subject is provided along with methods of using such a device. The device includes an inlet for a breath sample connected to a breath collection vessel. The device further includes a microprocessor associated with the inlet and breath collection vessel wherein the microprocessor is capable of automatically directing the breath sample to the breath collection vessel. The words “subject” and “patient” are used herein to refer to a human or animal from which a breath sample is being collected.

According to another aspect, an automated breath collection device is provided that includes a breath inlet with a breath sample reservoir connected thereto. A breath sample collection vessel is connected to the breath sample reservoir. The device further includes a valve associated with the breath sample reservoir and breath sample collection vessel. A microprocessor is associated with the breath collection vessel and the breath reservoir and is capable of opening the valve to automatically direct the breath sample from the breath reservoir to the breath collection vessel.

According to another aspect, a method of collecting breath samples is provided. The method includes the steps of having a subject breath into a breath collection device through an inlet until a desired amount of breath has been introduced into a breath sample reservoir and automatically conveying the breath exhaled from the subject from the breath sample reservoir to an airtight breath sample collection vessel.

According to another aspect, a method of automatically and interactively collecting breath samples from a subject is provided. The method includes directing a subject to exhale into the breath collection device through a breath inlet via a communication element in the breath collection device, capturing the breath sample in a breath sample reservoir, conveying the breath sample from the reservoir to a breath sample collection vessel, and purging the breath sample reservoir chamber.

According to another aspect, there is provided a system for collecting samples of breath. The system includes a device that includes a breath inlet adapted to be connected to a breath collection vessel, a microprocessor, and a cartridge that is releasably insertable into the device and adapted to hold the breath collection vessel.

According to another aspect, a cartridge for housing breath collection vessels is provided. The cartridge includes a frame releasably insertable into a breath collection device. The frame is adaptable to secure a breath collection vessel. The cartridge further includes a code positioned on the cartridge so that when the cartridge is inserted into the device, the code is read by a code reading element and one of a plurality of programs stored in a microprocessor of the device is chosen and activated.

### BRIEF DESCRIPTION OF THE DRAWINGS

FIG. 1 is a top view of an embodiment of the breath collection device according to the invention.

FIG. 2 is a bottom view of the embodiment of the breath collection device of FIG. 1 with the casing removed.

FIG. 3 is a perspective view of an embodiment of a breath collection vessel cartridge of the invention.

FIG. 4 is a perspective view of an alternate embodiment of the cartridge shown in FIG. 3.

FIG. 5 is an isolated view of the breath sample capture structures of the breath collection device shown in FIGS. 1, 2, 3, and 4.

FIG. 6 is schematic flow chart of the operation of the breath collection device of the invention.

### DETAILED DESCRIPTION

The following detailed description should be read with reference to the drawings, in which like elements in different drawings are numbered identically. The drawings, which are not necessarily drawn to scale, depict selected embodiments and are not intended to limit the scope of the invention. Several forms of the invention will be shown and described, and other forms will now be apparent to those skilled in the art. It will be understood that embodiments shown in drawings and described are merely for illustrative purposes and are not intended to limit the scope of the invention as defined in the claims that follow.

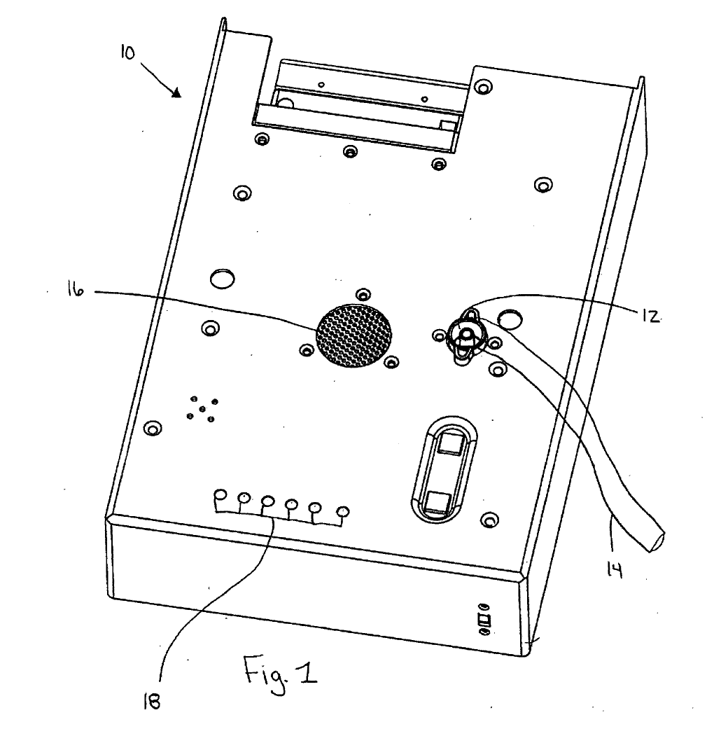

FIG. 1 is a top view of an automated breath collection device 10 according to an embodiment of the invention. The device is constructed so that it is of a size making it easily portable. In one embodiment, the device is approximately the size of a standard ream of notebook paper. The portable size, as well as the ease of use, as will be described in more detail below, allows flexibility in the manner in which the device can be used. For example, the small size is desirable since space is at a premium at health care facilities. Since the breath test is a non-invasive procedure, it is not necessary to perform the test in an examination room. Therefore, to optimize efficiency in a clinic or other health care facility, a patient or subject undergoing the breath test can be moved out of the examination room to a waiting room or other comfortable environment for completion of the procedure. Additionally, the device may be equipped with a carry strap so that clinical personnel can easily transport and supervise the test procedure such as when administering the test to an infant. Finally, the device can be easily carried by the subject if the breath test protocol requires the test to be administered while the subject is in motion.

As can be seen in FIG. 1, the breath collection device 10 includes an inlet 12 for the breath sample to be captured. The inlet 12 is adapted to be connected to conduit 14 to direct breath expired from a patient into the device 10. As depicted, the conduit 14 may be a hollow flexible hose. However, any structure that connects a subject's mouth to the device is suitable. For example, the conduit may be a hollow, rigid structure, similar to a drinking straw. The inlet 12 is depicted in FIG. 1 positioned on the top of the breath collection device. However, the placement of the inlet is of no import and may be positioned at any location along the top, sides, or bottom of the device.

The conduit 14 is adaptable to be connected to a nasal canula (not shown), a breathing tube (not shown) a mouth piece (not shown), a face mask, or other means into which a subject may blow a sample of breath. In instances where the subject is an infant or animal, the conduit may be connected to an face mask sized to fit over the subject's nose and mouth when a breath sample is to be collected. Alternately, the subject may exhale directly into the conduit 14.

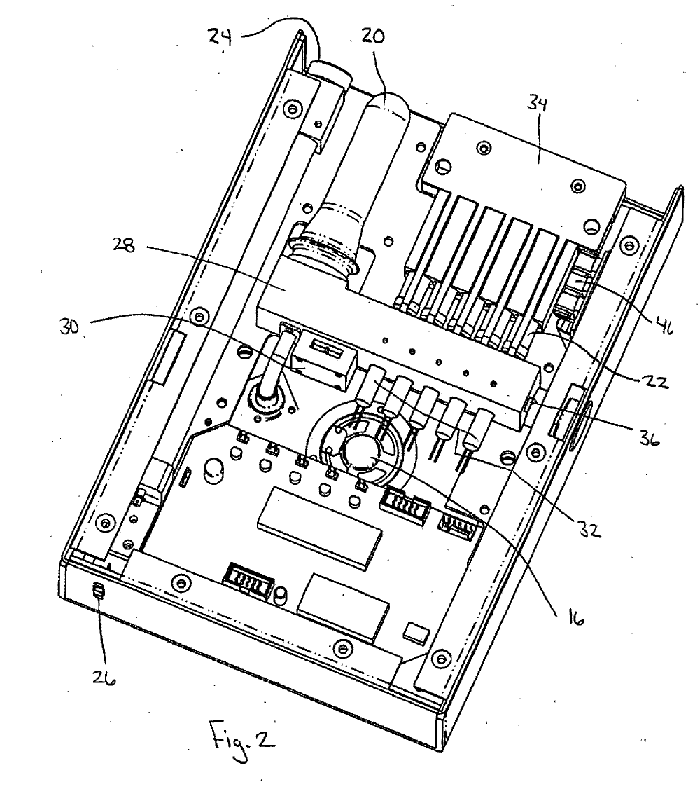

The breath collection device 10 may also include a speaker 16 located so that sounds emitted therefrom are audible to a user of the device 10. As depicted in FIG. 1, the speaker 16 is conveniently located on the top of the device 10 so that the user can easily discern test procedure instructions, which will be described further with respect to FIG. 2. An earplug adaptation may also be used if the speaker is inconvenient. Additionally, a visual display, such as, for example, a liquid crystal LED display, may be employed to provide visual instruction to a user in addition to or in place of audio instruction.

The device 10, in one embodiment, further includes a plurality of light emitting diodes (LED) 18 for indicating the status of various functions. For example, a LED may be provided to indicate that the device has been powered up. Additionally, the device may include a plurality of LEDs 18 that correspond to each of a plurality of collection vessels (discussed further with respect to FIGS. 2, 4, and 5). The device 10 may be programmed so that the LED is activated once a breath sample has been delivered to the corresponding breath collection vessel. Other functions that may be displayed via a LED include, without limitation, battery level, whether a breath collection procedure has begun, and when the collection procedure is complete. Light emitting diodes are only one means of visually indicating the status of various device functions. Other means may also be used. A microprocessor may also be used to store and retrieve data such as the actual sequence of events that took place during the breath collection procedure, including, but not limited to, time at which each sample was collected and whether a sample was collected in response to an instruction. That data may then be communicated to the user audibly or visually.

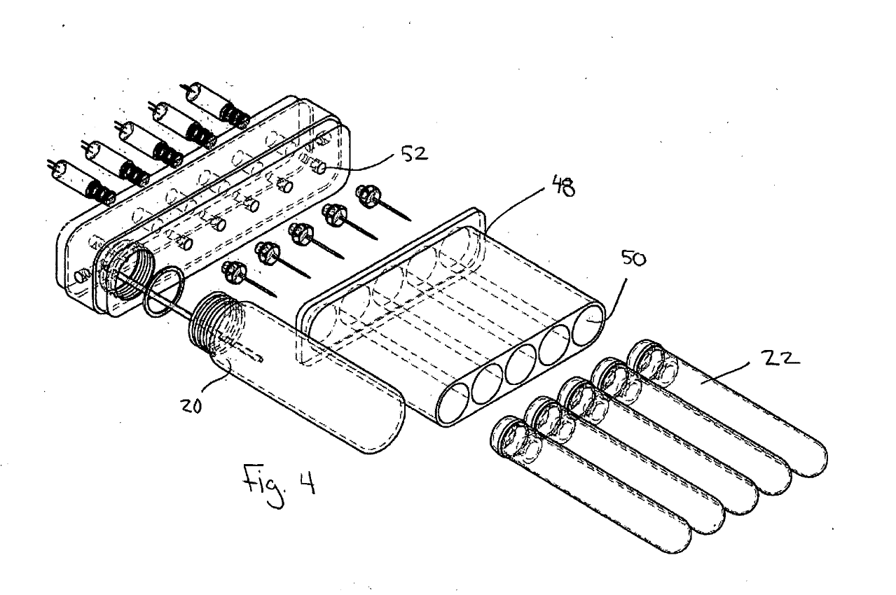

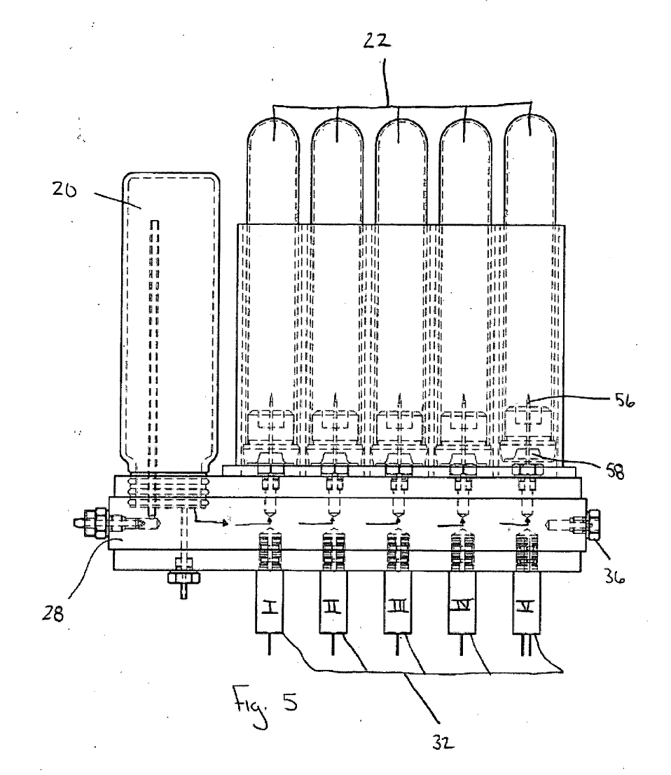

FIG. 2 is a bottom view of the embodiment of the breath collection device 10 depicted in FIG. 1 with the bottom panel removed to reveal internal components. Breath inlet 12 can be seen connected to a breath sample reservoir 20. Breath entering the device through the inlet 12 travels to the reservoir 20 and is diverted to a breath sample collection vessel 22 (described in more detail with respect to FIGS. 4 and 5).

In the embodiment pictured, the breath collection device 10 includes a communication element 16, which in the figures is a speaker, for emitting collection procedure instructions to the user. The speaker 16 is coupled to a set of directions contained within the device. The set of directions may be programmed into voice chips or a microprocessor, or other media capable of storing directions to be transmitted through a speaker 16. In one embodiment, a voice chip, (not shown) programmed with the instructions for any particular collection procedure is connected to the speaker 16.

In one method of the invention, when the device is used to collect breath samples for the ¹³C-gastric emptying test, the subject is instructed, via the speaker 16 to blow into the inlet 12 for a baseline sample. The speaker (or visual display) then directs the patient to consume a labeled substrate, usually a food item, and at specified time intervals thereafter alerts the patient to introduce breath into the breath collection inlet. The instructions can be programmed in any language appropriate to the ethnic population for which it is used. For the visually impaired, instructions can be provided on Braille cards. For the hearing impaired, the instructions may be provided visually on a display provided on the device 10. In addition, the patient may be provided with a written set of instructions to be read before commencement of the collection procedure so that the patient is familiar with the operation of the device. The use of pre-programmed instructions and self-guided test administration frees up valuable clinical resources.

In accordance with another embodiment, breath collection parameters, such as number of samples to be collected and collection time points, are manually entered into the device. The parameters are selected according to the requirements of the particular diagnostic test being conducted. For example, if three samples, taken at 30 minute intervals are required for a particular test procedure, then the device 10 is programmed so that the subject is directed to blow into the inlet 12 three times with a 30 minute interval between each of the first and second and second and third breath collections.

To program breath collection procedure instructions, the device may include a microprocessor. The keyboard is connected to the microprocessor by means known to those skilled in the art. Alternatively, the microprocessor may be connected to an external personal computer so that parameters may be stored or entered on the personal computer and downloaded to the microprocessor located within the device 10 or by any other suitable means.

In one embodiment, the device 10 is powered by batteries so that the device is readily portable. The batteries may be housed in a battery holder 24. Battery power is desirable in situations where hospital or clinical personnel conduct the test in patient rooms or off-site facilities where power outlets may not be readily available or when the test must be carried out while the subject is in motion. Alternately, the device may be powered by direct current. For situations where alternating current is heavily relied upon, the device may be equipped with a portable battery charger. The batteries or direct current power source is connected to an on/off switch 26 for turning the device on and off.

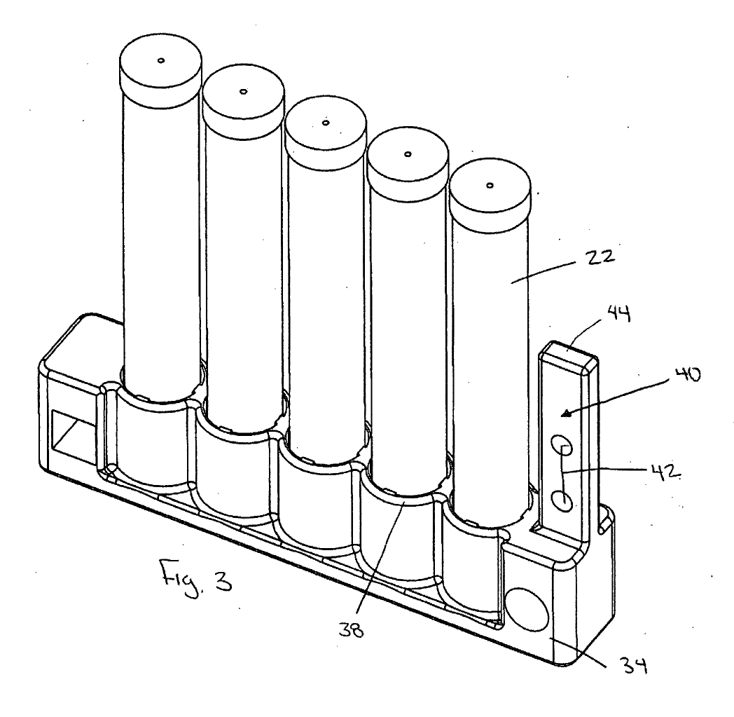

As breath enters the breath inlet 12, it travels to a breath reservoir 20 that is connected to an integrated valve manifold 28. The valve manifold 28 may be connected to a low pressure switch 30, flow sensor, pressure sensor, or other suitable device for detecting when a breath sample enters the device. The manifold 28 includes one or more electric valves 32 that direct the breath sample into one or more breath sample collection vessels 22 housed in a cartridge 34, which is described in greater detail with respect to FIG. 3.

The valve manifold 28 further includes an exhaust valve 36 for eliminating a breath sample from the device 10. In operation, the exhaust valve 36 is opened after a portion of a breath sample is directed to a collection vessel 22 and the remaining sample purged from the device 10 so that the next breath sample may be taken. The remnants of any sample can be fully evacuated by flushing a portion of the subsequent breath sample through the device 10. Alternately, a nitrogen gas supply (not shown) can be connected to the device 10 and a volume of the gas flushed through the system to eliminate the current sample retained in the device 10.

Referring to FIGS. 2 and 3, a cartridge 34 secures and retains breath sample collection vessels 22. The cartridge depicted in the FIGS. 2 and 3 includes five slots 38 to secure five collection vessels 22. The cartridge may be filled with as many as five collection vessels or as few as one, depending on the requirements of the test procedure to be administered for which the samples are being collected. As will be appreciated, the cartridge may be formed so as to secure as few as one or as many collection vessels as desired. If more than five breath samples are required, the cartridge holding the collection vessels filled with samples can be removed and replaced with a new cartridge housing fresh, empty collection vessels. Alternatively, the cartridge can be configured to house more than five vessels by utilizing additional slots 38.

In one embodiment, the cartridge 34 includes a machine-readable code 40. In one aspect, the code includes a series of one or more holes 42 formed in a tab 44 on the cartridge. The tab 44 is positioned on the cartridge so that when the cartridge 34 is inserted into the device 10 the one or more holes 42 formed in the tab 44 will align with a code reading element positioned within the device 10 such as the photo detectors 46 shown in FIG. 2. Other machine readable codes known in the art may be used with an appropriate code-reading element. In the embodiment shown, the tab 44 can accommodate up to three holes drilled in each of position 1, 2, and 3. In the figures, only two holes 42 are shown. The number and position of the holes 42 is detected by the photo detectors 46. The photo detectors are associated with the microprocessor and the code present on the cartridge can be used to choose which pre-programmed sequence should be activated for a test protocol associated with the code on the cartridge. For example, the microprocessor may have a plurality of programs stored within its memory, each program being designed to control the various electrical and mechanical components of the device through parameters specific for a desired breath collection protocol. Each program will be associated with a cartridge code. In this embodiment, the possible number of codes can be increased by increasing the number of holes formed in the tab and the corresponding photo detectors.

In another embodiment, the machine readable code is in the form of a bar code that may be read by a bar code reader (not shown) positioned in the device to be aligned with the inserted cartridge and wherein the bar code reader is connected to the microprocessor. Other non-machine readable coding systems may also be used to identify the breath samples to be collected in a particular cartridge. For example, the cartridge may be color coded so that when the user inserts the cartridge into the device, the user will manually select a particular testing protocol associated with a particular color.

The number of breath collection time points may vary from two, such as is required for the urea H. pylori test to five, such as is required for the ¹³C-Spirulina gastric emptying assessment test. Likewise, the duration of time between each breath sample may vary. Some test protocols requires 30 minute or less intervals whereas others require longer intervals, depending on for which test the breath sample will be used. To accommodate the varying breath collection procedures required for the variety of breath tests, the device will be associated with a microprocessor that includes a memory for storing the variety of breath collection procedures. The microprocessor may be part of the device or capable of being electronically connected to the device. As already described, the breath collection procedures of a particular protocol may be initiated by reading of the code 40 provided on the cartridge 34. Alternately, the device 10 may include a data entry keyboard, or be linkable to a personal computer so that breath collection procedures may be downloaded so that specific parameters or collection protocols can be manually entered or selected.

As discussed above, the codes, in one embodiment, can be associated with a specific color indicated on the cartridge, for example, the code for initiating the parameters for the ¹³C-gastric emptying test can be associated with red colored cartridges. Associating specific codes with colors allows the individual administering the breath collection procedure to quickly and easily identify the appropriate cartridge. Furthermore, the cartridge can be equipped with a bar code or other unique identifier that is associated with a particular patient to ensure that breath samples collected remain properly identified with the appropriate patient.

The cartridge 34 may be molded in plastic or other suitable material. For further convenience, the cartridge 34 can be configured so as to be adaptable to auto samplers attached to analytical instrumentation. Thus, once the samples are collected, the cartridge is removed and can be directly loaded in an analytical instrument, such as, for example, the autosampling module of a gas-isotope-ratio mass spectrometer without the need for removing the collection vessels 22.

In an alternative embodiment, shown in FIG. 4, the cartridge is not used. Instead, in this embodiment, the collection vessels 22 are held in arrangement by a guide block 48 that includes laterally spaced orifices 50. When the collection vessels 22 are inserted through the orifices 50 they are aligned with portages 52 in the valve manifold 28. The guide block 48 may be formed of molded plastic, metal or any other suitable material, by techniques well known to those skilled in the art and can include any number of orifices as desired.

The collection vessels may be of any size and shape depending on requirements of the specific test to be administered. For example, if smaller breath samples are acceptable, then the cartridge may be sized and shaped to retain a smaller breath sample. Conversely, a cartridge adapted to retain larger sample containers may be provided in instances where a larger volume of breath sample is required. The cartridge can be configured so as to accommodate varying sized collection vessels by means well known to those skilled in the art. The vessels 22 are desirably adapted to maintain an airtight seal so that the breath sample does not escape and to prevent impurities and air from the outside environment from contaminating and diluting the sample. The collection vessels shown in FIG. 3 are provided with sealing rubber stoppers 54. An example of a suitable collection container is the VACUTAINER® tube (Becton & Dickinson, Franklin Lakes, N.J. USA).

The cartridge 34, with the collection vessels 22 secured therein, as shown in FIG. 5, is positioned adjacent to the manifold 28 so that breath samples may be directed to the collection vessels 22 shown by the arrows. In the embodiment shown, the manifold includes five electric valves, numbered I-IV associated with five portages, numbered IP-IVP that correspond to and deliver breath samples to five collection vessels 22. The electric valves 32 are connected to and respond to a microprocessor that directs the opening and closing of the valves 32. Each valve 32 is connected to a needle 56 that punctures the septa 58 to deliver the breath sample to the collection vessel 22. Desirably, the needles are non-boring, so that the septa retains the air tight seal after the needle is withdrawn.

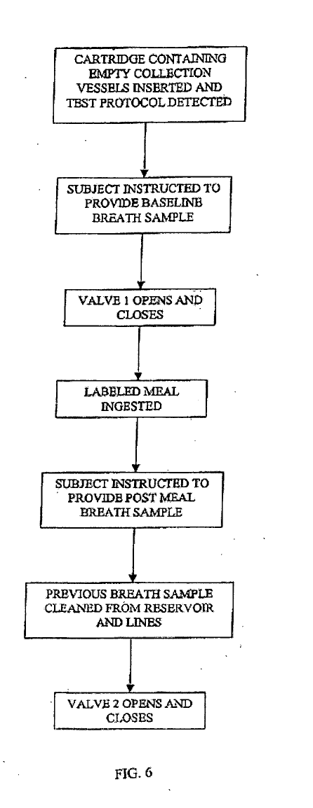

FIG. 6 is a schematic flow chart of the operation of the device 10. The operation of the device 10 will be described with respect to the breath collection procedures relevant to the ¹³C-gastric emptying test for illustrative purposes only. As already described, the device can be used to collect breath samples for any diagnostic test or purpose. The device is loaded with a cartridge containing five empty breath collection vessels. The cartridge may include a code for initiating the pre-loaded test parameters in the device or alternatively, the pre-loaded set of instructions can be manually selected. The pre-loaded instructions initiate the proper pre-programmed voice chip so that the subject is vocally directed through the breath collection procedure.

The first vocal command directs the patient to blow a breath into the breath inlet so that the device can collect a baseline sample. After the breath has entered the breath reservoir and is detected by a low pressure switch, a timer is activated and indicates when the patient may terminate blowing into the tube by an audible or visual or both, alert. This ensures that a volume of breath sufficient to clean the reservoir and lines as well as provide the required volume of sample is provided. The microprocessor opens valve 1, so that the breath sample is directed to the portage associated with that valve. The needle associated with that valve is activated and injects a portion of the breath sample into the collection vessel adjacent that the portage. The microprocessor desirably records the time at which the baseline sample was collected. A light emitting diode corresponding to the collection vessel number 1 is illuminated to indicate that vessel has been filled with a sample.

The breath chamber and lines are cleaned by a fraction of the subsequent breath sample blown into the device. Alternatively, the breath reservoir and lines may be connected to a nitrogen supply and a sufficient amount of nitrogen flushed through to eliminate all traces of the previous breath sample.

The subject is then directed to consume an edible substrate labeled with ¹³C. The device may include an indication button that the patient may push to confirm that the substrate has been consumed. The microprocessor may record the time taken to consume the substrate.

At pre-programmed time intervals, the subject is directed by voice command to input a breath sample that is directed, through the sequential opening of valves, to individual collection vessels. The time of each sample is recorded for subsequent confirmation that the test was performed accurately. Additionally, as each vessel is filled the light emitting diode on the face of the device is illuminated to indicate the status of each collection vessel.

At the conclusion of the test, the cartridge is removed from the device so that it may be analyzed by appropriate analytical instrumentation and appropriate personnel review the recorded information about the test details to confirm that the test was performed accurately.

While preferred embodiments of the present invention have been described, it should be understood that various changes, adaptations and modifications may be made therein without departing from the spirit of the invention and the scope of the appended claims.

1. An automated device for collecting samples of breath expired from a subject comprising:
an inlet for a breath sample;
a breath collection vessel connected to the inlet; and
a microprocessor associated with the inlet and breath collection vessel capable of automatically directing the breath sample to the breath collection vessel.

2. The device of claim 1, wherein the device is sized and shaped so as to be portable.

3. The device of claim 1, further comprising a valve associated with the breath collection vessel.

4. The device of claim 3, further comprising a valve manifold.

5. The device of claim 4, further comprising a plurality of breath collection vessels connected to the valve manifold.

6. The device of claim 1, further comprising a communication element.

7. The device of claim 6, wherein the communication element is a speaker.

8. The device of claim 7, wherein verbal breath collection procedure instructions capable of being transmitted through the speaker are stored in the microprocessor.

9. The device of claim 1, further comprising a breath capture mask associated with the breath sample inlet.

10. The device of 1, further comprising a nasal canula associated with the breath sample inlet.

11. The device of claim 1, further comprising a nitrogen gas supply associated with the breath sample inlet.

12. An automated breath collection device comprising;
a breath inlet;
a breath sample reservoir connected to the breath inlet;
a breath sample collection vessel connected to the breath sample reservoir;
a valve associated with the breath sample reservoir and breath sample collection vessel; and
a microprocessor associated with the breath collection vessel and the breath reservoir capable of opening the valve to automatically direct the breath sample from the breath reservoir to the breath collection vessel.

13. The device of claim 12, further comprising an exhaust valve connected to the breath sample reservoir.

14. The device of claim 12, further comprising a plurality of breath sample collection vessels and a plurality of valves, wherein each valve connects the breath sample reservoir to one of the breath sample collection vessels.

15. The device of claim 14, wherein the microprocessor is capable of sequentially opening the valves to deliver breath sample from the breath sample reservoir to one of the breath sample collection vessels.

16. The device of claim 12, further comprising a communication element associated with the microprocessor and wherein the microprocessor is capable of directing a subject to breath into the breath inlet according to a desired a breath collection procedure through the communication element.

17. The device of claim 12, further comprising a cartridge capable of holding the breath collection vessels.

18. The device of claim 17, wherein the cartridge comprises a machine readable code positioned on the cartridge so that when the cartridge is inserted into the device, the code is aligned with a code reading element associated with the microprocessor and wherein the code when read will activate the device to perform a preprogrammed breath collection procedure.

19. A method of collecting breath samples comprising the steps of;
having a subject breath into a breath collection device through an inlet until a desired amount of breath has been introduced into a breath sample reservoir; and
automatically conveying the breath exhaled from the subject from the breath sample reservoir to an airtight breath sample collection vessel.

20. The method of claim 19, wherein the breath collection device includes a communication element through which verbal instructions may be delivered and the method further includes directing the subject through the breath collection process using the communication element.

21. The method of claim 19, further including the step of cleaning the breath sample reservoir chamber.

22. The method of claim 21, wherein the breath sample reservoir is cleaned with nitrogen gas.

23. A method of automatically collecting breath samples from a subject comprising the steps of;
directing a subject to exhale into the breath collection device through a breath inlet via a communication element in the breath collection device;
capturing the breath sample in a breath sample reservoir;
conveying the breath sample from the reservoir to a breath sample collection vessel; and
cleaning the breath sample reservoir chamber.

24. A system for collecting samples of breath comprising:
a device including;
a breath inlet adapted to be connected to a breath collection vessel; and
a microprocessor; and
a cartridge releasably insertable into the device and adapted to hold the breath collection vessel.

25. The system of claim 24, wherein a valve is associated with the breath inlet and the breath collection vessel.

26. The system of claim 25, wherein the microprocessor controls the operation of the valve.

27. The system of claim 24, wherein the cartridge comprises a code that is positioned with respect to the cartridge so that when the cartridge is inserted into the device, a desired program stored in the microprocessor is initiated and opens the valve at specified time intervals.

28. The system of claim 24, wherein the device further includes a communication element that emits audible instructions to a user of the device.

29. A cartridge for housing breath collection vessels comprising:
a frame releasably insertable into a breath collection device, the frame adaptable to secure a breath collection vessel; and
a code positioned on the cartridge so that when the cartridge is inserted into the device, one of a plurality of programs stored in a microprocessor of the device is chosen and activated when the code is read by a code reading element.

30. The cartridge of claim 29, wherein the code comprises a bar code.

31. The cartridge of claim 29, wherein the code comprises holes formed in the cartridge frame.

32. The cartridge of claim 29, wherein the frame secures a plurality of breath collection vessels.
